(19) United States
(12) Plant Patent Application Publication
(10) Pub. No.: US 2006/0200881 P1
(43) Pub. Date: Sep. 7, 2006

(54) Variety of hydrangea plant named 'JON01'

(76) Inventor: Jos de Jong, Boskoop (NL)

Correspondence Address: FULWIDER PATTON LEE & UTECHT, LLP, 200 OCEANGATE, SUITE 1550, LONG BEACH, CA, 90802, US

(21) Appl. No.: 11/070,934

(22) Filed: Mar. 3, 2005

(51) Int. Cl. A01H 5/00

(52) U.S. Cl. PLT/250

(57) ABSTRACT

A new and distinct cultivar of hydrangea plant named ‘JON01’ characterized by its distinctive color of flowers.

### BACKGROUND OF THE NEW PLANT

The present invention relates to a new and distinct cultivar of Hydrangea, botanically known as Hydrangea L. macrophylla and further known by the varietal name ‘JON01’. This new variety is also sold under the Breeder's reference name LADY IN RED. The plant is used primarily as an ornamental. The variety ‘JON01’ has been asexually reproduced by cuttings. The novel flower color was consistent through several generations. The new variety was discovered in the summer of 2001 by the breeder Jos de Jong in the breeder's nursery, Gebr. De Jong Handelskwekerijen BV in Boskoop, The Netherlands. The parental cultivar is sold under the Breeder's reference name “Taube.”

### SUMMARY OF THE INVENTION

The following traits have been repeatedly observed and are determined to be among the unique characteristics of ‘JON01’. These characteristics distinguish ‘JON01’ as a new and distinct cultivar:
1. Different color of flowers, with the comparator (Taube) being dark pink to rosy-pink and this variety being dark rosy-red.

Plants of the cultivar ‘JON01’ have not been observed under all possible environmental and cultural conditions. The phenotype may vary somewhat with variations in environment such as temperature, light intensity, length of day, irrigation amount and frequency, and/or fertilizer rate without, however, any variance in genotype.

### BRIEF DESCRIPTION OF THE DRAWINGS

The invention is depicted in the accompanying colored photographs in which:

FIG. 1 shows a mature potted plant of the new variety;

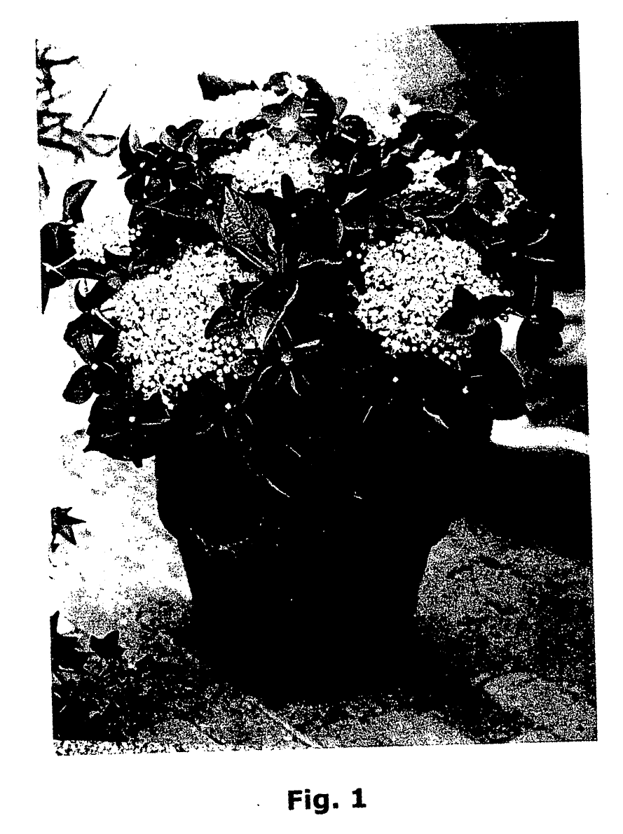

The photograph depicts the overall appearance of the new hydrangea showing the colors as true as it is reasonably possible to obtain in colored reproductions of this type. Colors in the photographs may differ slightly from the color values cited in the Detailed Botanical Description, which more accurately describe the actual colors.

### DETAILED BOTANICAL DESCRIPTION OF THE NEW PLANT

The following detailed description sets forth characteristics of the new cultivar.
Botanical Name: Hydrangea L. macrophylla.
Varietal Name: ‘JON01’.
Parentage: Mutation of “Taube”.
Propagation: Vegetatively by cuttings.
Inflorescence:
   Color midzone upper surface.—59B/60A (red-purple group).
Development:
   Resistance to disease.—Similar to other H. macrophylla varieties.

### GENERAL CHARACTERISTICS

‘JON01’ is a mutation of the taube and differs from taube by the color of the flowers, with the comparator being dark pink to rosy pink and the present invention characterized by flowers being dark rosy-red.

What is claimed is:

1. A new and distinct variety of hydrangea plant, substantially as shown and described.
